(19) United States
(12) Patent Application Publication
(10) Pub. No.: US 2024/0197812 A1
(43) Pub. Date: Jun. 20, 2024

(54) Methods of Treating Cardiac Injury

(71) Applicant: Proletariat Therapeutics, Inc., Andover, MA (US)

(72) Inventors: Emily See, East Melbourne (AU); James Wilkie, Dunstable, MA (US); Lakhmir Chawla, San Diego, CA (US); Rinaldo Bellomo, Ivanhoe East (AU)

(21) Appl. No.: 18/079,545

(22) Filed: Dec. 12, 2022

(51) Int. Cl. A61K 38/08; A61K 31/137; A61K 38/095; A61P 9/02

(52) U.S. Cl. CPC A61K 38/085; A61K 31/137; A61K 38/095; A61P 9/02

(57) ABSTRACT

Disclosed are methods of treating cardiac injury in a subject comprising administering a therapeutically effective amount of angiotensin II to the subject. Also disclosed are methods of treating catecholamine-associated cardiac injury in a subject comprising administering a therapeutically effective amount of angiotensin II to the subject. The subject can have cardiac injury, or the subject can be at risk of having cardiac injury.

### BACKGROUND

Vasodilatory shock is a life-threatening condition of circulatory failure that results in hypotension, reduced tissue perfusion, progressive organ failure, and an increased risk of death. Patients with vasodilatory shock are commonly treated with a vasopressor such as a catecholamine and/or vasopressin to act as vasoconstrictors to increase blood pressure.

Catecholamines are monoamine neurotransmitters having a catechol and a side-chain amine. Catecholamines include epinephrine (adrenaline), norepinephrine (noradrenaline) and dopamine. Catecholamines cause general physiological changes that prepare the body for physical activity (the fight-or-flight response). Some typical effects are increases in heart rate, blood pressure, blood glucose levels, and a general reaction of the sympathetic nervous system. Vasopressin is a polypeptide hormone that causes contraction of vascular and other smooth muscles and antidiuresis. The chemical name of vasopressin is Cyclo (1-6) L-Cysteinyl-L-Tyrosyl-L-Phenylalanyl-L-Glutaminyl-L-Asparaginyl-L-Cysteinyl-L-Prolyl-L-Arginyl-L-Glycinam. Administration of both catecholamines and vasopressin have been shown to be effective in many patients to maintain mean arterial pressure at a desired level. However, administration of both catecholamines and vasopressin can have toxic effects.

Excessive catecholamine exposure can adversely impact the function of several organs, including the heart. Dünser M W, et al., “Sympathetic overstimulation during critical illness: adverse effects of adrenergic stress,” J Intensive Care Med 24:293-316, 2009. Human patients exposed to exogenous norepinephrine administered as a therapeutic intervention have been reported to develop myocardial lesions. Histologic lesions indicating catecholamine-associated cardiotoxicity (e.g., contraction band necrosis, myocytolysis, and mononuclear cell infiltrate) can be demonstrated in almost all patients who die from septic shock and correlate with the total duration and dose of catecholamine therapy. Baroldi G. et al., “Myocardial contraction bands. Definition, quantification and significance in forensic pathology,” Int J Legal Med 115:142-151, 2001; Schmittinger C A, et al, “Histologic pathologies of the myocardium in septic shock: A prospective observational study,” Shock 39:329-335, 2013. Similarly, excessive vasopressin exposure can lead to cardiac injury in some patients. Indrambarya et al., “Low-dose vasopressin infusion results in increased mortality and cardiac dysfunction following ischemia-reperfusion injury in mice,” Crit. Care, 13(3), R98, 2009.

Angiotensin II is a naturally occurring peptide hormone that has been approved by the FDA for use as a vasoconstrictor to increase blood pressure in adults with septic or other distributive shock. GIAPREZA® Prescribing Information, 12/2021.

Angiotensin II has only been studied in patients with catecholamine-refractory vasodilatory shock. In the double-blind ATHOS-3 trial (Angiotensin II for the Treatment of High-Output Shock trial), adults with septic or other distributive shock who remained hypotensive despite fluid and vasopressor therapy were administered angiotensin II and 70% of patients achieved a MAP ≥75 mmHg or a ≥10 mmHg increase in MAP without change in baseline vasopressor therapy at 3 hours, compared to only 23% of subjects who received placebo (p<0.0001).

Chawla describes methods of administering angiotensin II to a subject having high output shock and undergoing treatment with a catecholamine, wherein the angiotensin II is effective to raise or maintain mean arterial pressure (MAP) of the subject, and reducing the dose of the catecholamine. U.S. Pat. No. 9,220,745. Chawla does not provide any evidence that the administration of angiotensin II will enable sufficient reduction of catecholamine administration to an extent sufficient to treat (e.g., mitigate, prevent) any catecholamine-associated toxicity, or in particular cardiac injury associated with catecholamine administration.

The Surviving Sepsis Campaign Guidelines Committee, which issues guidelines reflecting global consensus for the treatment of sepsis and septic shock, consistently recommends using norepinephrine (a catecholamine) as the first-line treatment for hypotension in adults with septic shock, followed by vasopressin as the second-line treatment. Since both catecholamines and vasopressin are known to cause cardiac injury, the primary first-line and second-line vasopressors are medications with a demonstrated capacity to cause cardiac injury.

Further, subarachnoid hemorrhage (SAH) is a devastating cerebrovascular condition, not only due to the effect of the initial hemorrhage, but also due to the complication of delayed cerebral ischemia (DCI). While hypertension facilitated by vasopressors is often initiated to prevent DCI, which vasopressor is most effective in improving outcomes is not known. Williams, et al., “Vasopressor treatment and mortality following nontraumatic subarachnoid hemorrhage: a nationwide electronic health record analysis,” Neurosurg. Focus, 48(5):E4 (May 1, 2020). During hypertensive therapy for post-subarachnoid hemorrhage (SAH) symptomatic vasospasm, norepinephrine is commonly used to reach target blood pressures. Concerns over aggravation of vasospasm with norepinephrine exist. Delayed cerebral ischemia (DCI) post-subarachnoid hemorrhage (SAH) poses significant challenges to the treating physician. Current standard therapy consists of hypertensive directed measures in order to ensure preservation of cerebral blood flow to risk territories. Zeiler, et al., “Norepinephrine as a potential aggravator of symptomatic cerebral vasospasm: Two cases and Argument for Milrinone therapy,” Case Reports in Critical Care (2014).

As such, there is a need in the art for methods to prevent or treat cardiac injury including cardiac injury resulting from catecholamine and/or vasopressin. In addition, there is a need in the art for methods to increase blood pressure in patients in need of being held in a hypertensive state. The present invention describes such methods.

### BRIEF SUMMARY

The present invention provides methods for treating (including mitigating and preventing) cardiac injury in a subject comprising administering angiotensin II to the subject. In such methods, the subject may be at risk and is in need of a vasopressor (e.g., a catecholamine and/or vasopressin, i.e., one or more catecholamines alone or in combination with vasopressin, or vasopressin alone) to increase blood pressure, or the subject may be at risk and is currently taking a vasopressor (e.g., a catecholamine and/or vasopressin) to increase blood pressure.

Included in the invention are methods of treating cardiac injury in a subject who is experiencing cardiac injury. In such methods the administration of angiotensin II can act to mitigate cardiac injury. Also disclosed are methods of treating cardiac injury in a subject who does not have cardiac injury but is at risk of suffering cardiac injury. In such methods the administration of angiotensin II can act to prevent cardiac injury.

Disclosed are methods of treating cardiac injury in a subject, wherein the subject has catecholamine-associated cardiac injury, or risk of cardiac injury, e.g., due to the use of exogenous vasopressor, such as a catecholamine and/or vasopressin. Also disclosed are methods of treating cardiac injury in a subject wherein the cardiac injury or risk of cardiac injury relates to the subject being currently administered or prospectively administered a vasopressor, such as a catecholamine and/or vasopressin. Also disclosed are methods of treating cardiac injury in a subject, wherein the cardiac injury or risk of cardiac injury relates to the subject having a medical condition which causes endogenous release of catecholamines. In such methods the subject may be experiencing cardiac injury, or the subject may not have cardiac injury but is at risk of suffering cardiac injury.

In some aspects, the subjects being treating according to the disclosed methods are being treated with, or are in need of being treated with, a vasopressor such as a catecholamine and/or vasopressin to increase and maintain blood pressure. In some aspects, the administration of a vasopressor such as a catecholamine and/or vasopressin is discontinued or is adjusted, for example, the dose and/or dosing frequency of vasopressor (e.g., catecholamine) is reduced. In some aspects, the administration of angiotensin II enables the discontinuation and/or reduction of the vasopressor administration such that treatment (e.g., mitigation and/or prevention) of cardiac injury is achieved.

In other aspects, the disclosed methods can include a step of determining whether the subject to be treated has suffered cardiac injury, e.g., as a result of being administered a catecholamine and/or vasopressin. In some embodiments, the present invention provides methods wherein the cardiac injury is identified based on the presence of blood troponin. In other embodiments, the present invention provides methods wherein the administration of angiotensin II prevents increases in blood troponin and/or prevents cardiac injury and/or prevents worsening of cardiac injury (e.g., mitigation). In yet other embodiments, the present invention provides methods wherein the administration of angiotensin II to a subject reduces blood troponin and/or reduces cardiac injury. In some aspects, the administration of angiotensin II allows for a reduction in, or elimination of, the administration of a catecholamine and/or vasopressin.

Also disclosed are methods of treatment wherein the administration of angiotensin II to a subject suffering cardiac injury provides a clinically significant reduction in blood troponin following angiotensin II administration.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION

The disclosed methods may be understood more readily by reference to the following detailed description of particular embodiments and the Example included therein and to the Figures and their previous and following descriptions.

It is to be understood that the disclosed methods are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a dose” includes a plurality of such doses, reference to “the dose” is a reference to one or more doses and equivalents thereof known to those skilled in the art, and so forth.

As used herein, the term “subject” or “patient” refers to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses, and pigs; laboratory animals such as mice, rats, and guinea pigs; rabbits; fish; reptiles; zoo and wild animals). Typically, “subjects” are animals, including mammals such as humans and primates, and the like.

As used herein, the term “treating” refers to partially or completely alleviating, ameliorating, relieving, mitigating, preventing, delaying onset of, inhibiting, or slowing progression of, reducing severity of, and/or reducing incidence of cardiac injury. Treatment can be administered to a subject who does not exhibit signs of cardiac injury and/or to a subject who exhibits signs of cardiac injury.

As used herein, “preventing” is meant to mean minimize the chance that a subject who has an increased susceptibility for developing a disease, disorder or condition will develop the disease, disorder, or condition (e.g., cardiac injury, catecholamine-associated cardiac injury). For example, prevent as used herein can mean minimize the chance that a subject who is at risk of cardiac injury will develop it.

As used herein, the terms “administering” and “administration” refer to any method of providing a disclosed composition (e.g., angiotensin II) to a subject. Such methods are well known to those skilled in the art and include, but are not limited to: oral administration, transdermal administration, administration by inhalation, nasal administration, topical administration, intravaginal administration, ophthalmic administration, auricular administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The phrase “cardiac injury” means the state of having myocardial injury or damage. The myocardial injury can be acute or chronic. There is a broad spectrum of myocardial injury. Myocardial injury may be related to acute myocardial ischemia (e.g., atherosclerotic plaque disruption with thrombosis) or it may be related to acute myocardial ischemia because of oxygen supply/demand imbalance (e.g., reduced myocardial perfusion or increased myocardial oxygen demand). Other causes of myocardial injury include cardiac conditions (e.g., heart failure, myocarditis, cardiomyopathy, Takotsubo syndrome, coronary revascularization, catheter ablation, defibrillator shocks, cardiac contusion) or systemic conditions (e.g., sepsis, chronic kidney disease, stroke, pulmonary embolism, infiltrative disease, chemotherapeutic agents, critical illness, strenuous exercise). Myocardial injury includes myocardial necrosis, myocardial infarction (heart attack), cardiomyopathy, myocardial ischemia, and blunt cardiac injury.

The phrase “troponin” means the troponin complex comprising three regulatory proteins (troponin C, troponin I, and troponin T). Troponin I (cardiac troponin I, denoted cTnI) and troponin T (cardiac troponin T, denoted cTnT) are used as diagnostic and prognostic indicators for myocardial injury. Diagnostic criteria for raised troponin indicating myocardial infarction is currently set by the WHO at a threshold of 2 μg or higher.

Cardiac troponin T and I are measured by immunoassay methods. Roche Diagnostics distributes its proprietary cTnT and a variety of diagnostic companies make cTnI immunoassay methods available on many different immunoassay platforms. The 99th percentile cutoff for cardiac troponin T (cTnT) is 0.01 ng/mL. The reference range for the high sensitivity troponin T is a normal <14 ng/L, borderline of 14-52 ng/L, and elevated of >52 ng/L.

The term “angiotensin II” may refer to Asp-Arg-Val-Tyr-Ile-His-Pro-Phe [SEQ ID NO: 1] also called 5-isoleucine angiotensin II. SEQ ID NO: 1 is an octa-peptide naturally present in humans and other species, such as equines, hogs, etc. Isoleucine may be substituted by valine to result in 5-valine angiotensin II, Asp-Arg-Val-Tyr-Val-His-Pro-Phe [SEQ ID NO: 2]. Other angiotensin II analogues such as [Asn1-Phe4]-angiotensin II [SEQ ID NO: 3], hexapeptide Val-Tyr-Ile-His-Pro-Phe [SEQ ID NO: 4], nonapeptide Asn-Arg-Val-Tyr-Tyr-Val-His-Pro-Phe [SEQ ID NO: 5], [Asn′-Ileu5-Ileu8]-angiotensin II [SEQ ID NO: 6], [Asn1-Ileu5-Ala8]-angiotensin II [SEQ ID NO: 7], and [Asn1-diiodoTyr4-Ileu5]-angiotensin II [SEQ ID NO: 8] may also be used. Angiotensin II may be synthesized, for example, by solid phase peptide synthesis to incorporate modifications, such as C-terminal amidation. C-terminal acetate groups may also be added. Fragments of angiotensin II which retain the activity of angiotensin II may also be used, for example angiotensin II fragments wherein an N terminal amino acid is deleted, or angiotensin II fragments in which a C terminal is deleted (e.g., Ang III, Ang IV and Ang II (1-7)). The term “angiotensin II,” without further specificity, is intended to refer to any of these various forms, as well as combinations thereof.

The term “catecholamine,” as used herein, refers to dopamine, norepinephrine, epinephrine, phenylephrine, ephedrine and their prodrugs, structural analogs, or derivatives that induce similar physiological effects in humans, e.g., raise mean arterial pressure in healthy human subjects. In certain embodiments, the catecholamine may be dopamine, norepinephrine, epinephrine, ephedrine, or phenylephrine.

The term “pharmaceutically acceptable salt,” as used herein, represents those salts which are, within the scope of sound medical judgment, suitable for use. The physicochemical and biological properties of drug substances are greatly affected by their salt forms. The choice of a particular salt formulation is based on numerous factors such as the drug substance chemistry, intended dosage form, pharmacokinetics, and pharmacodynamics. The selection of a counterion is based on the degree of ionization of the acidic or basic functional groups that are present in the drug. Consideration is made with respect to the drug substance contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66: 1-19, 1977, and in Pharmaceutical Salts: Properties, Selection, and Use, (Eds. P. H. Stahl and C. G. Wermuth), Wiley-VCR, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

An “effective amount” of a composition is that amount of composition which is sufficient to provide a beneficial effect to the subject to which the composition is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. For example, a therapeutically effective amount of angiotensin II is an amount that is sufficient to increase blood pressure and/or to treat cardiac injury. Such therapeutic effect on cardiac injury may be determined by evaluating the effect on cardiac troponin levels.

“Optional” or “optionally” means that the subsequently described event, circumstance, or material may or may not occur or be present, and that the description includes instances where the event, circumstance, or material occurs or is present and instances where it does not occur or is not present.

“Vasopressor” means a drug (e.g., anti-hypotensive agent, vasopressor agent, or pressor” that tends to raise low blood pressure, e.g., by causing the constriction of blood vessels. Vasopressors include, e.g., catecholamines, vasopressin and combinations thereof.

Ranges may be expressed herein as from “about” one particular value, and/or to “about” another particular value. When such a range is expressed, also specifically contemplated, and considered disclosed, is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers, or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Angiotensin II Therapeutics

The methods of the invention involve administration of angiotensin II to a subject.

Angiotensin II is a peptide hormone naturally produced by the body. Angiotensin II regulates blood pressure via vasoconstriction and sodium reabsorption. Hemodynamic effects of angiotensin II administration have been the subject of numerous clinical studies, demonstrating significant effects on systemic and renal blood flow (Harrison-Bernard, L. M., The renal renin-angiotensin system. Adv Physiol Educ., (2009) 33(4): p. 270-74). Angiotensin II is a hormone produced by the renin-angiotensin-aldosterone system (RAAS) that modulates blood pressure via regulation of vascular smooth muscle tone and extracellular fluid homeostasis. Angiotensin II mediates its effects on the vasculature by inducing vasoconstriction and sodium retention. In addition to its systemic effects, angiotensin II has a pronounced effect on the efferent arterioles of the kidney, maintaining glomerular filtration when blood flow is decreased. Angiotensin II also regulates sodium reabsorption in the kidney by stimulating Na+/H+ exchangers in the proximal tubule and inducing the release of aldosterone and vasopressin (Harrison-Bernard, L. M., The renal renin-angiotensin system. Adv. Physiol. Educ., 2009. 33(4): p. 270-4).

The sequence of angiotensin II used in the compositions and methods disclosed herein may be homologous to the sequences of angiotensin II described above. In certain aspects, the invention includes isolated, synthetic, or recombinant amino acid sequences that are at least 80%, 85%, 90%, 95%, 97%, 98%, 99% or 100% identical to SEQ ID NO: 1, 2, 3, 4, 5, 6, 7, and/or 8. Any such variant sequences may be used in place of an angiotensin II as described in the preceding paragraph.

In some aspects, the angiotensin II may be selected from 5-valine angiotensin II, 5-valine angiotensin II amide, 5-L-isoleucine angiotensin II, and 5-L-isoleucine angiotensin II amide, or a pharmaceutically acceptable salt thereof, preferably manufactured under current good manufacturing conditions (cGMP). In some aspects, the composition may include different forms of angiotensin II in different percentages, e.g., a mixture of hexapeptide and nonapeptide angiotensin. The composition comprising angiotensin II may be suitable for parenteral administration, e.g., for injection or intravenous infusion.

Similarly, an angiotensin II therapeutic may be used as any suitable salt, deprotected form, acetylated form, deacetylated form, and/or prodrug form of the above-mentioned peptides, including pegylated forms of the peptides or conjugates as disclosed in U.S. Pat. No. 7,666,408 (incorporated by reference). The term “prodrug” refers to any precursor compound which is able to generate or to release the above-mentioned peptide under physiological conditions. Such prodrugs may be larger peptides which are selectively cleaved in order to form the peptide of the invention. For example, in some aspects, the prodrug may be angiotensin I or its homologues that may result in angiotensin II by the action of certain endogenous or exogenous enzymes. Further prodrugs include peptides with protected amino acids, e.g., having protecting groups at one or more carboxylic acid and/or amino groups. Suitable protecting groups for amino groups are the benzyloxycarbonyl, t-butyloxycarbonyl (BOC), fluorenylmethyloxycarbonyl (FMOC), formyl, and acetyl or acyl group. Suitable protecting groups for the carboxylic acid group are esters such as benzyl esters or t-butyl esters. The present invention also contemplates the use of angiotensin II and/or precursor peptides having amino acid substitutions, deletions, additions, the substitutions and additions including the standard D and L amino acids and modified amino acids, such as, for example, amidated and acetylated amino acids, wherein the therapeutic activity of the base peptide sequence is maintained at a pharmacologically useful level.

In some aspects the angiotensin II is synthetic human angiotensin II having a chemical name of L-Aspartyl-L-arginyl-L-valyl-L-tyrosyl-L-isoleucyl-L-histidyl-L-prolyl-L-phenylalanine, acetate salt. Molecular formula: C₅₀H₇₁N₁₃O₁₂·(C₂H₄O₂)ₙ; (n=number of acetate molecules; theoretical n=3), Average molecular weight: 1046.2 (as free base).

### C. Doses of the Therapeutically Effective Substance

In general, angiotensin II increases blood pressure. The composition including the angiotensin therapeutic (e.g., angiotensin II) can be administered at a dose and/or at a rate sufficient to increases blood pressure and/or to achieve a target blood pressure. For example, a patient may be coupled to a monitor that provides continuous, periodic, or occasional measurements of mean arterial pressure (MAP).

In some aspects the composition including the angiotensin therapeutic (e.g., angiotensin II) can be administered at a dose and/or rate sufficient to enable the reduction in dose of another vasopressor being administered or about to be administered, such as a catecholamine, while still achieving the desired increase in blood pressure and/or target blood pressure. For example, the amount of catecholamine being administered can be reduced or eliminated such that cardiac injury, or risk of cardiac injury, associated with catecholamine administration can be reduced or avoided.

In some aspects the composition including the angiotensin therapeutic (e.g., angiotensin II) can be administered at a dose and/or at a rate sufficient to treat cardiac injury, e.g., to prevent cardiac injury from occurring, to prevent cardiac injury from increasing, to prevent an increase in levels of biomarkers associated with cardiac injury, and/or to maintain and/or reduce the levels of biomarkers associated with cardiac injury. In some aspects the composition including the angiotensin therapeutic (e.g., angiotensin II) can be administered at a dose and/or at a rate sufficient to achieve a target blood pressure and to treat cardiac injury.

The dose of angiotensin II can be administered at a rate of from about 0.25 ng/kg/min to about 100 ng/kg/min, e.g., from about 5 ng/kg/min to about 20 ng/kg/min, from about 10 ng/kg/min to about 50 ng/kg/min, from about 20 ng/kg/min to about 40 ng/kg/min, from about 0.25 ng/kg/min to about 20 ng/kg/min, from about 0.25 ng/kg/min to about 10 ng/kg/min, from about 0.25 ng/kg/min to about 5 ng/kg/min, from about 1.25 ng/kg/min to about 20 ng/kg/min, about 1.25 ng/kg/min to about 10 ng/kg/min, or from about 1.25 ng/kg/min to about 5 ng/kg/min. In embodiments of the invention, the dose is about 0.25 ng/kg/min, about 0.5 ng/kg/min, about 1 ng/kg/min, about 1.25 ng/kg/min, about 1.5 ng/kg/min, about 2 ng/kg/min, about 2.5 ng/kg/min, about 3 ng/kg/min, about 3.5 ng/kg/min, about 4 ng/kg/min, about 4.5 ng/kg/min, about 5 ng/kg/min, about 5.5 ng/kg/min, about 6 ng/kg/min, about 7.5 ng/kg/min or about 10 ng/kg/min.

In some embodiments of the invention, the starting dose of angiotensin II is 20 ng/kg/min, e.g., administered via continuous intravenous infusion. The angiotensin II dose can be titrated upwards and/or downwards as needed to achieve or maintain a target blood pressure and/or to treat cardiac injury. Titration of angiotensin II dose can enable a simultaneous decrease in the dose of vasopressor, e.g., catecholamine and/or vasopressin. In some embodiments, titration of angiotensin II dose enables elimination of vasopressor, e.g., catecholamine and/or vasopressin, administration.

The dose of angiotensin II can be titrated upwards by increments of up to 15 ng/kg/min, wherein the dose does not exceed 80 ng/kg/min during the first 3 hours of treatment. The dose may also be down-titrated every 5 to 15 minutes by increments of up to 15 ng/kg/min. Maintenance doses should not exceed 40 ng/kg/min. Doses as low as 1.25 ng/kg/min may be used.

The dose administration can last from about 0.25 hours to about 120 hours, e.g., from about 1 hour to about 7 hours, 2 hours to about 6 hours, or about 3 hours to about 5 hours. The therapeutic regimen can be started within, e.g., 1 hour, 2 hours, 4 hours, 6 hours, 12 hours, 24 hours, 48 hours, or 72 hours, after the onset of acute symptoms.

### D. Modes of Administration

Administration of angiotensin II can be by any convenient route, e.g., intravenous (using either a bolus or by a steady infusion), intramuscular, intra-osseos, subcutaneous or inhalation.

The compositions of the invention can be administered in a variety of conventional ways. In some aspects, the compositions of the invention are suitable for parenteral administration. These compositions may be administered, for example, intraperitoneally, intravenously, intra-arterially, intrarenally, or intrathecally. In some aspects, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

### E. Subject Population

The method of the invention involves administering angiotensin II to a subject. The subject may have hypotension, normotension, or hypertension depending on the clinical context and disease state.

The subject typically is at risk (e.g., is hypotensive) and is in need of treatment with a vasopressor, e.g., one or more catecholamines, vasopressin, or a combination thereof, to raise the subject's blood pressure. Such subjects may be critically ill patients with shock, such as high output shock or distributive shock (septic shock), or shock from cardiac arrest or cardiogenic shock. Subjects may also have acute kidney injury, hepato-renal syndrome and/or variceal bleeding. Subjects may also require intentional hypertension and require vasopressor therapy to increase blood pressure, e.g., to increase intracranial perfusion pressure so as to avoid delayed cerebral ischemia. For example, the subject may have suffered from stroke, subarachnoid hemorrhage, or traumatic brain injury.

One aspect of the invention is a method comprising administering to a subject, wherein the subject has cardiac injury. A subject having cardiac injury may be identified based on the presence of a detectable amount of troponin in blood. A subject having cardiac injury may also be identified based on the presence of an elevated amount of troponin present in the blood. Diagnosis of cardiac injury is often based on detecting the presence of Troponin I (cardiac troponin I, denoted cTnI) and/or troponin T (cardiac troponin T, denoted cTnT). Myocardial injury is defined as being present when blood levels of cTn are increased above the 99th percentile upper reference limit (URL). The injury may be acute, as evidenced by a newly detected dynamic rising and/or falling pattern of cTn values above the 99th percentile URL, or chronic, in the setting of persistently elevated cTn levels. Cardiac troponin T and I are measured by immunoassay methods. Roche Diagnostics distributes its proprietary cTnT and a variety of diagnostic companies make cTnI immunoassay methods available on many different immunoassay platforms. The 99th percentile cutoff for cardiac troponin T (cTnT) is 0.01 ng/mL. The reference range for the high sensitivity troponin T is a normal <14 ng/L, borderline of 14-52 ng/L, and elevated of >52 ng/L. A subject having cardiac injury may also be identified based on echocardiography, e.g., by the presence of regional wall motion abnormalities (RWMA) visible in an echocardiogram. Alternatively, a subject having cardiac injury may be identified based on a combination of the presence of e.g., elevated cardiac troponin and by the presence of regional wall motion abnormalities (RWMA).

In some aspects the subject having cardiac injury may be a subject whose cardiac injury is associated with, e.g., is a result of, being administered or having been administered a catecholamine (e.g., catecholamine-induced cardiac injury) and/or vasopressin. Such a subject may be administered or may have been administered a catecholamine and/or vasopressin at a dose level that creates cardiac injury or may be administered or may have been administered a catecholamine and/or vasopressin for a duration long enough to create a cardiac injury. In yet other aspects, the subject having cardiac injury may be a subject who is in need of being administered a catecholamine and/or vasopressin, or who is in need of being administered a catecholamine at a dose level that creates cardiac injury or may be administered or may have been administered a catecholamine and/or vasopressin for a duration long enough to create a cardiac injury.

In some aspects the subject is one having septic or other distributive shock and having cardiac injury. For example, the subject may have been administered a catecholamine and/or vasopressin to treat septic or other distributive shock and may have catecholamine-induced and/or vasopressin-induced cardiac injury.

Another aspect of the invention is a method comprising administering to a subject not having cardiac injury. A subject not having cardiac injury may be identified based on the absence of detectable amount of troponin in blood. A subject not having cardiac injury may also be identified based on the absence of an elevated amount of troponin present in the blood, e.g., the absence of Troponin I (cardiac troponin I, denoted cTnI) and/or troponin T (cardiac troponin T, denoted cTnT), or a subject having blood levels of cTn less than or equal to the 99th percentile upper reference limit (URL). A subject not having cardiac injury may also be identified based on echocardiography, e.g., by the absence of regional wall motion abnormalities (RWMA) visible in an echocardiogram. Alternatively, a subject having cardiac injury may be identified based on a combination of the absence of e.g., elevated cardiac troponin and by the absence of regional wall motion abnormalities (RWMA).

In some aspects, the subject not having cardiac injury may be at risk of cardiac injury. Subjects having a risk of cardiac injury may be subjects having cardiac risk factors including hypertension, diabetes mellitus, heart failure, coronary artery disease, atrial fibrillation/flutter, and cerebrovascular disease, advanced age, history of smoking, history of pulmonary or kidney disease and/or diagnosis with COVID-19. Alternatively, or in addition, the subject having a risk of cardiac injury may be a subject who is being administered or has been administered a catecholamine. Such a subject may be administered or may have been administered a vasopressor, such as a catecholamine and/or vasopressin, at a dose level that creates cardiac injury or may be administered or may have been administered a vasopressor, such as a catecholamine and/or vasopressin, for a duration long enough to create a cardiac injury. In yet other aspects, the subject having a risk of cardiac injury may be a subject who is in need of a catecholamine and/or vasopressin, or who is in need of being administered a catecholamine and/or vasopressin at a dose level that creates cardiac injury or may be administered or may have been administered a catecholamine and/or vasopressin for a duration long enough to create a cardiac injury.

In some aspects the subject not having cardiac injury is one having septic or other distributive shock. For example, the subject may have been administered a catecholamine and/or vasopressin to treat septic or other distributive shock and may be at risk of having catecholamine-induced and/or vasopressin-induced cardiac injury.

In some aspects the subject having cardiac injury or not having cardiac injury is a subject having a medical condition which causes endogenous release of catecholamines. For example, the subject may have neuroendocrine tumors (e.g., pheochromocytomas), neuroblastoma, convulsive status epilepticus, cerebrovascular diseases such as ischemic stroke, brain hemorrhage and subarachnoid hemorrhage, or the subject may experience a surge in catecholamine after craniotomy.

### F. Methods Related to the Administration of Angiotensin II

One aspect of the invention is a method of treatment comprising administering to a subject having cardiac injury a therapeutically effective dose of angiotensin II. In some aspects, the therapeutically effective amount of angiotensin II prevents further progression of the cardiac injury. In such embodiment, the prevention of cardiac injury may be achieved by reducing the subject's need for treatment with another vasopressor such as a catecholamine and/or vasopressin.

Another aspect of the invention is a method of treatment comprising administering to a subject having cardiac injury a therapeutically effective dose of angiotensin II, wherein the subject's cardiac injury is associated with catecholamine and/or vasopressin administration. In some aspects, the therapeutically effective amount of angiotensin II prevents further progression of the cardiac injury, e.g., by enabling for a reduction in catecholamine and/or vasopressin dose while maintaining the subjects acceptable blood pressure.

In some embodiments, the subject is undergoing standard-of-care treatment with a vasopressor, e.g., a catecholamine (e.g., epinephrine, norepinephrine, dopamine, phenylephrine, ephedrine) and/or vasopressin at or prior to the time the angiotensin II is administered. These agents may be administered at a dose which is equivalent to at least 0.05 mcg/kg/min of the catecholamine, norepinephrine (a norepinephrine equivalent dose, NED). Typical equivalent doses are:

| | | |
|---|---|---|
| Drug | Dose | Norepinephrine Equiv. |

| | | | | |
|---|---|---|---|---|
| Epinephrine | 0.1 | mcg/kg/min | 0.1 | mcg/kg/min |
| Norepinephrine | 0.1 | mcg/kg/min | 0.1 | mcg/kg/min |
| Dopamine | 15 | mcg/kg/min | 0.1 | mcg/kg/min |
| Phenylephrine | 1.0 | mcg/kg/min | 0.1 | mcg/kg/min |
| Vasopressin | 0.04 | U/min | 0.1 | mcg/kg/min |

In some embodiments, the dose of the vasopressor, e.g., catecholamine (e.g., epinephrine, norepinephrine, dopamine, phenylephrine, ephedrine) and/or vasopressin at or prior to the time the angiotensin II is administered exceeds a typical equivalent dose.

In some aspects, the subject has previously been administered a conventional vasopressor (e.g., a catecholamine and/or vasopressin) and the method can further include adjusting (e.g., reducing or titrating downward) the dose of a conventional vasopressor (e.g., a catecholamine and/or vasopressin) as compared to the dose that the subject was taking prior to the angiotensin II administration. Such adjustment can involve reducing the amount of catecholamine being administered or can involve reducing the dosing frequency of the catecholamine. In some embodiments, the administration of catecholamine is discontinued at the time of administering the angiotensin II.

In other aspects, the subject is in need of treatment with a vasopressor (e.g., a catecholamine and/or vasopressin) and the methods can comprise administering angiotensin II in the absence of a catecholamine and/or vasopressin to achieve a target blood pressure. The methods can also comprise administering angiotensin II in combination with a conventional vasopressor (e.g., a catecholamine and/or vasopressin) and then adjusting (e.g., reducing or titrating downward or discontinuing) the dose of the conventional vasopressor (e.g., a catecholamine and/or vasopressin) as compared to the starting dose of the conventional vasopressor. In addition, or alternatively, the methods can comprise administering angiotensin II in combination with a conventional vasopressor (e.g., a catecholamine and/or vasopressin) and then adjusting (e.g., increasing or titrating upward) the dose of the angiotensin II. Titration of the angiotensin II and/or conventional vasopressor dose can be carried out to achieve the desired blood pressure control as well as to treat or prevent cardiac injury.

In some aspects, the methods comprising administration of angiotensin II are effective to raise the blood pressure of the subject to a mean arterial pressure (MAP) of about 65 mm Hg or above, or about 70 mm Hg or above, or about 75 mm Hg or above, or about 80 mm Hg, or about 85 mm Hg or above, or about 90 mm Hg or above, or about 95 mm Hg or above, or about 100 mm Hg or above. In still other aspects, the methods comprising administration of angiotensin II are effective to raise the blood pressure of the subject to a mean arterial pressure (MAP) above about 65 mm Hg, and which is effective to reduce the dose of the catecholamine and/or vasopressin required to maintain a MAP above about 65 mm Hg to the equivalent of less than about 0.05-0.10 mcg/kg/min NED, or are effective to raise the blood pressure of the subject to a mean arterial pressure (MAP) above about 70 mm Hg, and which is effective to reduce the dose of the catecholamine and/or vasopressin required to maintain a MAP above about 70 mm Hg to the equivalent of less than about 0.05-0.10 mcg/kg/min NED, or are effective to raise the blood pressure of the subject to a mean arterial pressure (MAP) above about 75 mm Hg, and which is effective to reduce the dose of the catecholamine and/or vasopressin required to maintain a MAP above about 75 mm Hg to the equivalent of less than about 0.05-0.10 mcg/kg/min NED, or are effective to raise the blood pressure of the subject to a mean arterial pressure (MAP) above about 80 mm Hg, and which is effective to reduce the dose of the catecholamine and/or vasopressin required to maintain a MAP above about 80 mm Hg to the equivalent of less than about 0.05-0.10 mcg/kg/min NED, or are effective to raise the blood pressure of the subject to a mean arterial pressure (MAP) above about 85 mm Hg, and which is effective to reduce the dose of the catecholamine and/or vasopressin required to maintain a MAP above about 85 mm Hg to the equivalent of less than about 0.05-0.10 mcg/kg/min NED, or are effective to raise the blood pressure of the subject to a mean arterial pressure (MAP) above about 90 mm Hg, and which is effective to reduce the dose of the catecholamine and/or vasopressin required to maintain a MAP above about 90 mm Hg to the equivalent of less than about 0.05-0.10 mcg/kg/min NED, or are effective to raise the blood pressure of the subject to a mean arterial pressure (MAP) above about 95 mm Hg, and which is effective to reduce the dose of the catecholamine and/or vasopressin required to maintain a MAP above about 95 mm Hg to the equivalent of less than about 0.05-0.10 mcg/kg/min NED, or are effective to raise the blood pressure of the subject to a mean arterial pressure (MAP) above about 100 mm Hg, and which is effective to reduce the dose of the catecholamine and/or vasopressin required to maintain a MAP above about 100 mm Hg to the equivalent of less than about 0.05-0.10 mcg/kg/min NED.

In some embodiments the subject has elevated blood troponin levels (e.g., increased high sensitivity cardiac troponin, Troponin I, or the like) at the time of the administration of angiotensin II. In some aspects of these embodiments the administration of the therapeutically effective amount of angiotensin II to the subject prevents further increases in the amount of blood troponin in the subject. In other aspects of these embodiments the administration of the therapeutically effective amount of angiotensin II to the subject reduces further increases in the amount of blood troponin in the subject (e.g., compared to the increase that would be observed in the absence of angiotensin administration). In yet other embodiments the administration of the therapeutically effective amount of angiotensin II to the subject has the effect of reducing the amount of blood troponin in the subject.

Thus, in some aspects of the invention, the method further comprises a step of determining the blood troponin level of the subject prior to administration of angiotensin II. In some aspects of the invention, the method further comprises a step of determining the blood troponin level of the subject after administration of angiotensin II. In other aspects of the invention, the method further comprises comparing the blood troponin level of the subject prior to administration of angiotensin II to the blood troponin level of the subject after administration of angiotensin II. Further, in some aspects, the method comprises a step of adjusting the dose of the angiotensin II based on the subjects blood pressure, usage of a catecholamine, and the level of blood troponin determined following administration of angiotensin II. For example, in some embodiments, the dose of angiotensin II is titrated upward when the level of blood troponin determined following administration of angiotensin II exceeds a predetermined threshold level of 99% URL. Other considerations for adjusting the dose of the angiotensin II may include the subject's blood pressure and usage of catecholamine.

In some embodiments the subject having catecholamine-associated cardiac injury has elevated blood troponin levels (e.g., increased high sensitivity cardiac troponin, Troponin I, or the like) at the time of the administration of angiotensin II. In some aspects of these embodiments the administration of the therapeutically effective amount of angiotensin II to the subject prevents further increases in the amount of blood troponin in the subject. In other aspects of these embodiments the administration of the therapeutically effective amount of angiotensin II to the subject reduces further increases in the amount of blood troponin in the subject (e.g., compared to the increase that would be observed in the absence of angiotensin administration). In yet other embodiments the administration of the therapeutically effective amount of angiotensin II to the subject reduces the amount of blood troponin in the subject.

Thus, in some aspects of the invention, the method further comprises a step of determining the blood troponin level of the subject prior to administration of angiotensin II. In some aspects of the invention, the method further comprises a step of determining the blood troponin level of the subject after administration of angiotensin II. In other aspects of the invention, the method further comprises comparing the blood troponin level of the subject prior to administration of angiotensin II to the blood troponin level of the subject after administration of angiotensin II. Further, in some aspects, the method comprises a step of adjusting the dose of the angiotensin II based on the subject's blood pressure, the dose of catecholamine and level of blood troponin determined following administration of angiotensin II. For example, in some embodiments, the dose of angiotensin II is increased or titrated upward when the level of blood troponin determined following administration of angiotensin II is greater than or equal to the amount of troponin present before administering angiotensin II.

In other aspects, the methods of the invention include each of the following methods.
1. A method of treating cardiac injury in a subject comprising administering a therapeutically effective amount of angiotensin II to a subject in need thereof.
2. The method of claim 1, wherein the subject has cardiac injury.
3. The method of any one of claims 1-2, wherein the subject has increased blood troponin.
4. The method of claim 3, wherein the blood troponin is a high sensitivity cardiac troponin (hs-cTn).
5. The method of claims 3 or 4, wherein the blood troponin is Troponin I.
6. The method of any one of claims 3-5, wherein increased blood troponin is blood troponin levels above the 99^{th} percentile upper reference limit.
7. The method of any one of claims 1-6, wherein the subject has wall motion abnormalities as determined by echocardiography.
8. The method of any of claims 1-7, wherein the therapeutically effective amount of angiotensin II prevents further progression of the cardiac injury.
9. The method of claim 1, wherein the subject does not have cardiac injury.
10. The method of claims 1 and 9, wherein the administration of angiotensin II allows for reduction of the amount of catecholamine and/or vasopressin needed to be administered to the subject.
11. The method of any of claims 9-10, wherein the therapeutically effective amount of angiotensin II prevents cardiac injury.
12. A method of treating catecholamine-associated or vasopressin-associated cardiac injury in a subject comprising administering angiotensin II to a subject in need thereof.
13. The method of claim 12, wherein the subject has cardiac injury.
14. The method of claim 13, wherein the subject has increased blood troponin.
15. The method of claim 14, wherein the blood troponin is a high sensitivity cardiac troponin (hs-cTn).
16. The method of any one of claims 14-15, wherein the blood troponin is Troponin I.
17. The method of any one of claims 14-16, wherein increased blood troponin is blood troponin levels above the 99^{th} percentile upper reference limit.
18. The method of any one of claims 12-17, wherein the administration of angiotensin II is effective to prevent increase in blood troponin.
19. The method of any one of claims 1-18, wherein the subject is taking or is in need of taking a catecholamine, vasopressin, or a combination thereof.
20. The method of claim 19, wherein the administration of angiotensin II allows for reduction of the amount of catecholamine and/or vasopressin being administered.
21. The method of claim 19, wherein the administration of angiotensin II allows for discontinuation of the administration of catecholamine and/or vasopressin.
22. The method of any one of claims 12-21, wherein the therapeutically effective amount of angiotensin II prevents further progression of the cardiac injury.
23. A method of treating catecholamine-associated or vasopressin-associated cardiac injury in a subject wherein the subject does not have cardiac injury, comprising administering angiotensin II to a subject in need thereof.
24. The method of claim 23, wherein the subject is taking or is in need of taking a catecholamine, vasopressin, or a combination thereof.
25. The method of any one of claims 23-24, wherein the therapeutically effective amount of angiotensin II prevents catecholamine-associated and/or vasopressin-associated cardiac injury.
26. The method of any one of claims 23-25, wherein the administration of angiotensin II allows for reduction of the amount of catecholamine and/or vasopressin needing to be administered to the subject.
27. A method of treating catecholamine-associated and/or vasopressin-associated cardiac injury in a subject who has been previously administered a catecholamine and/or vasopressin, comprising the steps of (1) administering angiotensin II to the subject and (2) discontinuing administration of the catecholamine and/or vasopressin.
28. A method of treating catecholamine associated cardiac injury comprising administering angiotensin II to a subject receiving catecholamine and/or vasopressin at an initial dose, comprising the steps of (1) administering angiotensin II to the subject and (2) administering catecholamine and/or vasopressin to the subject at a dose that is lower than the initial dose.
29. The method of claim 27 or 28, wherein the subject has suffered cardiac injury.
30. The method of claim 29, wherein the angiotensin II prevents further progression of the cardiac injury.
31. The method of claims 27 or 28, wherein the subject has not suffered cardiac injury.
32. The method of claim 31, wherein the angiotensin II prevents cardiac injury.
33. The method of any one of claims 28-32, wherein two or more doses of catecholamine and/or vasopressin are administered to the subject after the initial dose.
34. The method of claim 33, wherein each dose of catecholamine and/or vasopressin is lower than the previous dose.
35. The method of any one of claims 28-45, wherein the angiotensin II and the catecholamine and/or vasopressin are administered in separate compositions.
36. The method of any one of claims 28-45, wherein the angiotensin II and the catecholamine and/or vasopressin are administered in a single composition.
37. The method of any one of claims 12 to 36, wherein the catecholamine is norepinephrine.
38. The method of any one of claims 1-37, further comprising measuring the amount of troponin present in a sample obtained from the subject before administering angiotensin II.
39. The method of any one of claims 1-37, further comprising (1) measuring the amount of troponin present in a sample obtained from the subject before administering angiotensin II and (2) measuring the amount of troponin present in a sample obtained from the subject after administering angiotensin II.
40. The method of claim 39, wherein angiotensin II can be increased in a subject if the amount of troponin present in a sample obtained from the subject after administering angiotensin II is greater than or equal to the amount of troponin present in a sample obtained from the subject before administering angiotensin II.
41. The method of any one of claims 1-40, wherein the administration of angiotensin II allows for reduction in the amount of catecholamine and/or vasopressin being administered such that there is a clinically significant reduction in blood troponin.
42. A method of increasing blood pressure in a subject in need of being held in a hypertensive state comprising these steps of administering angiotensin II.
43. The method of claim 42 wherein the increase in blood pressure is needed to increase intracranial perfusion pressure.
44. The method of any one of claims 42-43 wherein the subject has suffered from stroke, subarachnoid hemorrhage, or traumatic brain injury.
45. The method of any one of claims 1-44, wherein angiotensin II is administered via intravenous infusion.
46. The method of claim 45, wherein angiotensin II is administered via continuous intravenous infusion.
47. The method of any one of claims 1-46, wherein angiotensin II is administered at a rate of about 1 ng/kg/min to 20 ng/kg/min.
48. The method of claim 47, further comprising titrating the rate upward to a rate of about 30 ng/kg/min to about 40 ng/kg/min.
49. The method of any one of claims 1-48, wherein the administration of angiotensin II is effective to raise the blood pressure of the subject to a mean arterial pressure (MAP) of about 65 mm Hg or above.
50. The method of any one of claims 1-49, wherein the administration of angiotensin II is effective to raise the blood pressure of the subject to a mean arterial pressure (MAP) above about 65 mm Hg, and which is effective to reduce the dose of the catecholamine and/or vasopressin required to maintain a MAP above about 65 mm Hg to the equivalent of less than about 0.05-0.10 mcg/kg/min NED.

### G. Combination Therapies

Disclosed are methods of treating cardiac injury in a subject comprising administering a therapeutically effective amount of angiotensin II alone or in combination with a catecholamine. In some aspects the angiotensin II and the catecholamine are administered in separate compositions. In other aspects the angiotensin II and the catecholamine are co-formulated. In various methods of the invention, the angiotensin II and the catecholamine may be administered, together or independently, intravenously, intramuscularly, subcutaneously or by inhalation. Separate administration of the angiotensin II and the catecholamine is particularly advantageous in methods wherein the dose of angiotensin II is going to be titrated so as to achieve a target blood pressure and to achieve a reduction and/or elimination of catecholamine administration and treatment of cardiac injury.

### H. Pharmaceutical Compositions

Suitable formulations (pharmaceutical compositions) for administering a drug (e.g., angiotensin II and/or one or more catecholamines and/or vasopressin) will depend on the mode of administration. For example, formulations adapted for parenteral administration may comprise a sterile aqueous preparation, preferably isotonic with the blood of the recipient. This aqueous preparation may be formulated according to known methods using suitable dispersing or wetting agents and suspending agents. The preparation also may be a sterile injectable solution or suspension in a diluent or solvent, for example as a solution with mannitol, 1,3-butanediol, water, Ringer's solution, and isotonic sodium chloride solution, which are exemplary acceptable diluents. Sterile, fixed oils may be employed as a solvent or suspending medium. Bland fixed oils, including synthetic mono or di-glycerides, and fatty acids, such as oleic acid, may also be used. Most of the agents described herein are commercially available and can be obtained readily from commercial sources.

The pharmaceutical compositions of the present invention may also contain diluents, fillers, salts, buffers, stabilizers, solubilizers, and other materials well known in the art. The term “pharmaceutically acceptable carrier” refers to a non-toxic carrier that may be administered to a patient, together with a therapeutically effective substance (such as angiotensin II) of this invention, and which does not destroy the pharmacological activity of the therapeutically effective substance. The term “pharmaceutically acceptable” means a non-toxic material that does not interfere with the effectiveness of the biological activity of the active ingredient(s). The characteristics of the carrier will depend on the route of administration. The term “excipient” refers to an additive in a formulation or composition that is not a pharmaceutically active ingredient. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to empirically determine which excipients, if any, to include in the compositions of the invention. Excipients of the invention may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some aspects, it may be beneficial to include a pharmaceutically acceptable carrier in the compositions of the invention.

In some aspects, it may be beneficial to include a solubilizing agent in the compositions of the invention. Solubilizing agents may be useful for increasing the solubility of any of the components of the formulation or composition, including a therapeutically effective substance (e.g., angiotensin II and/or catecholamine) or an excipient. The solubilizing agents described herein are not intended to constitute an exhaustive list, but are provided merely as exemplary solubilizing agents that may be used in the compositions of the invention. In certain aspects, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

In some aspects, it may be beneficial to adjust the pH of the compositions by including a pH-adjusting agent in the compositions of the invention. Modifying the pH of a formulation or composition may have beneficial effects on, for example, the stability or solubility of a therapeutically effective substance, or may be useful in making a formulation or composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions of the invention. pH-adjusting agents may include, for example, acids and bases. In some aspects, a pH-adjusting agent includes, but is not limited to, acetic acid, hydrochloric acid, phosphoric acid, sodium hydroxide, sodium carbonate, and combinations thereof.

The pH of the compositions of the invention may be any pH that provides desirable properties for the formulation or composition. Desirable properties may include, for example, therapeutically effective substance (e.g., angiotensin II) stability, increased therapeutically effective substance retention as compared to compositions at other pHs, and improved filtration efficiency. In some aspects, the pH of the compositions of the invention may be from about 3.0 to about 9.0, e.g., from about 5.0 to about 7.0. In particular aspects, the pH of the compositions of the invention may be 5.5±0.1, 5.6±0.1, 5.7±0.1, 5.8±0.1, 5.9±0.1, 6.0±0.1, 6.1±0.1, 6.2±0.1, 6.3±0.1, 6.4±0.1, or 6.5±0.1.

In some aspects, it may be beneficial to buffer the pH by including one or more buffers in the compositions. In certain aspects, a buffer may have a pKa of, for example, about 5.5, about 6.0, or about 6.5. One of skill in the art would appreciate that an appropriate buffer may be chosen for inclusion in compositions of the invention based on its pKa and other properties. Buffers are well known in the art.

Accordingly, the buffers described herein are not intended to constitute an exhaustive list, but are provided merely as exemplary buffers that may be used in the compositions of the invention. In certain aspects, a buffer may include one or more of the following: Tris, Tris HCl, potassium phosphate, sodium phosphate, sodium citrate, sodium ascorbate, combinations of sodium and potassium phosphate, Tris/Tris HCl, sodium bicarbonate, arginine phosphate, arginine hydro-chloride, histidine hydrochloride, cacodylate, succinate, 2-(N-morpholino)ethanesulfonic acid (MES), maleate, bis-tris methane, phosphate, carbonate, and any pharmaceutically acceptable salts and/or combinations thereof.

In some aspects, it may be beneficial to include a surfactant in the compositions of the invention. Surfactants, in general, decrease the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

In some aspects, it may be beneficial to include a tonicity-adjusting agent in the compositions of the invention. The tonicity of a liquid composition is an important consideration when administering the composition to a patient, for example, by parenteral administration. Tonicity-adjusting agents, thus, may be used to help make a formulation or composition suitable for administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In some aspects, it may be beneficial to include a stabilizing agent in the compositions of the invention. Stabilizing agents help increase the stability of a therapeutically effective substance in compositions of the invention. This may occur by, for example, reducing degradation or preventing aggregation of a therapeutically effective substance. Without wishing to be bound by theory, mechanisms for enhancing stability may include sequestration of the therapeutically effective substance from a solvent or inhibiting free radical oxidation of the anthracycline compound. Stabilizing agents are well known in the art. Accordingly, the stabilizing agents described herein are not intended to constitute an exhaustive list, but are provided merely as exemplary stabilizing agents that may be used in the compositions of the invention. Stabilizing agents may include, but are not limited to, emulsifiers and surfactants.

The compositions of the invention can be administered in a variety of conventional ways. In some aspects, the compositions of the invention are suitable for parenteral administration. These compositions may be administered, for example, intraperitoneally, intravenously, intra-arterially, intrarenally, or intrathecally. In some aspects, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The following examples are included to illustrate the invention, but the invention should not be understood to be limited to these exemplified embodiments.

### Example

A prospective observational study was conducted to evaluate the efficacy, safety feasibility of using angiotensin II as a primary vasopressor for treatment of critically ill patients with vasodilatory shock.

Study Population: A total of 40 patients receiving angiotensin II were matched to 80 control patients who received conventional vasopressors (norepinephrine, vasopressin, metaraminol, epinephrine, or combinations thereof). The patients were adult patients (≥18 years) with vasodilatory shock admitted to an intensive care unit who had received less than 24 hours of vasopressor therapy and had an arterial, central venous, and indwelling bladder catheter in situ. The mean patient age was 63 years (SD 14 years), two-thirds of patients were male, and the median APACHE III score was 65 (IQR 49 to 80). Baseline characteristics of the patient cohort are presented in Table 1. There were no significant differences between groups in terms of demographics, clinical characteristics, illness severity, baseline vasopressor type and dose, physiological parameters, or biochemistry.

TABLE 1
Demographic and clinical characteristics of critically ill patients
receiving angiotensin II or conventional vasopressors as first
line therapy for the treatment of vasodilatory shock.

| | Angiotensin II | | Control | | |
|---|---|---|---|---|---|
| | (N = 40) | | (N = 80) | | P value |
| Male sex - no. (%) | 28 | (70) | 51 | (64) | 0.50 |
| Age, years - mean (SD) | 62 | (12) | 63 | (14) | 0.64 |
| Weight, kg - mean (SD) | 84 | (26) | 88 | (35) | 0.47 |
| Cause of shock - no. (%) | | | | | 0.27 |
| Sepsis | 24 | (60) | 44 | (55) | |
| Pancreatitis | 2 | (5) | 0 | (0) | |
| Post-operative | 6 | (15) | 13 | (16) | |
| Multifactorial | 4 | (10) | 9 | (11) | |
| Other | 4 | (10) | 14 | (18) | |
| Illness severity - median (IQR) | | | | | |
| Apache II score | 16 | (13, 21) | 17 | (13, 23) | 0.47 |
| Apache III score | 64 | (52, 78) | 65 | (48, 82) | 0.93 |
| ANZROD score | 0.07 | (0.03, 0.21) | 0.09 | (0.04, 0.32) | 0.25 |
| Mechanical ventilation - no. (%) | 26 | (65) | 55 | (69) | 0.68 |
| CRRT - no. (%) | 5 | (12) | 10 | (12) | 1.00 |
| RAAS inhibitor use - no. (%) | | | | | 0.15 |
| Nil | 26 | (65) | 53 | (66) | 0.94 |
| ACE | 4 | (10) | 9 | (11) | |
| ARB | 10 | (25) | 18 | (22) | |
| Time from ICU admission to | 14 | (21) | 11 | (14) | 0.27 |
| intervention, hours - mean (SD) | | | | | |
| Time from vasopressor to intervention, | 7 | (7) | 5 | (6) | 0.12 |
| hours - mean (SD) | | | | | |
| Vasopressor use at intervention start - | | | | | |
| no. (%) | | | | | |
| Norepinephrine | 35 | (88) | 72 | (90) | 0.68 |
| Vasopressin | 8 | (20) | 16 | (20) | 1.00 |
| Metaraminol | 4 | (10) | 9 | (11) | 0.84 |
| Epinephrine | 1 | (2) | 8 | (10) | 0.14 |
| Vasopressor dose - median (IQR) | | | | | |
| Norepinephrine, mcg/min | 12 | (7, 18) | 10 | (5, 20) | 0.51 |
| Vasopressin, units/min | 0.04 | (0.02, 0.04) | 0.04 | (0.04, 0.04) | 0.13 |
| Metaraminol, mcg/min | 85 | (55, 150) | 80 | (70, 80) | 0.81 |
| Epinephrine, mcg/min | 5 | (5, 5) | 5 | (4, 9) | 0.84 |
| Physiology - median (IQR) | | | | | |
| Heart rate, bpm | 94 | (80, 105) | 87 | (70, 98) | 0.11 |
| Systolic blood pressure, mmHg | 101 | (95, 110) | 108 | (99, 115) | 0.13 |
| Mean arterial pressure, mmHg | 73 | (65, 75) | 71.50 | (67, 77) | 0.71 |
| Cardiac index, L/min/m² | 2.7 | (2.2, 3.6) | 2.6 | (2.2, 3.1) | 0.65 |
| Biochemistry - median (IQR) | | | | | |
| Sodium, mmol/L | 138 | (134, 140) | 136 | (134, 139) | 0.12 |
| Creatinine, μmol/L | 117 | (80, 173) | 116 | (83, 156) | 0.75 |
| Albumin, g/L | 28 | (24, 31) | 27 | (24, 31) | 0.78 |
| Bilirubin, μmol/L | 16 | (10, 40) | 15 | (8, 39) | 0.46 |
| INR | 1.3 | (1.1, 1.5) | 1.3 | (1.2, 1.6) | 0.49 |
| Standard base excess, mmol/L | −3 | (−6, −1) | −3 | (−7, 0) | 0.78 |
| Lactate, mmol/L | 2.3 | (1.3, 3.6) | 2.2 | (1.5, 5.0) | 0.15 |

Abbreviations: ACE, angiotensin converting enzyme; ANZROD, Australian and New Zealand Risk of Death; ARB, angiotensin receptor blocker, CRRT, continuous renal replacement therapy; ICU, intensive care unit; IQR, interquartile range; INR, international normalized ratio; RAAS, renin angiotensin aldosterone system; SD, standard deviation;

Vasodilatory shock was defined by a mean arterial pressure <65 mmHg in the presence of sufficient volume resuscitation and preserved cardiac output, based on either a cardiac index ≥2.3 L/min/m², a central venous oxygen saturation ≥70%, or clinician judgement. Patients were excluded if they had kidney failure (i.e., chronic hemodialysis, peritoneal dialysis or a baseline estimated glomerular filtration rate <15 mL/min/1.73 m²) or were diagnosed with arterial (acute coronary syndrome, ischemic stroke, or mesenteric ischemia) or venous thrombosis (deep vein thrombosis or pulmonary embolism) within the preceding 6 months. Patients were also excluded if they had a contraindication to chemical thromboprophylaxis in the first 48 hours. The mean patient age was 63 (SD 14) years, two-thirds of patients were male, and the median APACHE III score was 65 (IQR 49 to 80). There were no significant differences between the angiotensin II group and the conventional vasopressor group in terms of demographics, clinical characteristics, illness severity, vasopressor type and use, physiological parameters, or biochemistry.

Intervention: Infusions of angiotensin II were initiated to deliver a dose between 5 and 10 ng/kg/min. The dose was adjusted every 5 minutes according to a protocolized titration scheme to achieve a mean arterial pressure (MAP) of ≥65 mmHg (dose range 1.25 to 40 ng/kg/min). During the adjustment period, infusions of other vasopressor agents were weaned and discontinued where possible. Where the MAP was inadequate despite the maximum dose of angiotensin II, additional vasopressor agents were prescribed at the discretion of the clinician. The use of vasopressin (dose range 0.01 to 0.04 IU/min) was preferred over the use of norepinephrine (dose range 0 to 40 mcg/min). Angiotensin II was continued until resolution of shock, death, or for a maximum of 7 days. If ongoing vasopressor therapy was required after this time, norepinephrine was utilized.

Control: The control arm included critically ill patients with vasodilatory shock who received conventional vasopressors. To address confounding by indication and other sources of bias arising from the use of non-randomized data, a propensity score was estimated for the likelihood of treatment with angiotensin II to match patients in the intervention arm to their nearest neighbor in the control arm using a 1:2 ratio without replacement. Propensity scores were generated based on age, sex, APACHE II score, serum creatinine level, vasopressor type and dose at enrollment, and time from ICU admission and initiation of any vasopressor to initiation of angiotensin II.

Primary Outcome: The primary outcome was peak serum creatinine level at 7 days, censored at ICU discharge. Serum creatinine level was measured every 4-8 hours via a blood gas analyzer using the enzymatic method (ABL800 FLEX, Radiometer Medical). Patients who received CRRT during the first 7 days were not included in the primary analysis.

Secondary Outcomes: Secondary renal outcomes included new or progressive AKI, creatinine trajectory, the requirement for CRRT, time to CRRT liberation, and the development of a major adverse kidney event at day 7 (MAKE7). New AKI was defined and staged according to the Kidney Disease Improving Global Outcomes (KDIGO) criteria as a rise in serum creatinine of ≥26.5 μmol/L in 48 hours or as a ≥50% increase in serum creatinine from baseline within 7 days. Progressive AKI was defined as an increase in AKI severity by at least one KDIGO stage. MAKE7 was a composite of death, dialysis dependence, and a persistent 50% increase in serum creatinine level. Other secondary outcomes included all-cause mortality (ICU, hospital, 28- and 90-day), physiological parameters (heart rate [HR], systolic blood pressure [SBP], mean arterial pressure [MAP], diastolic blood pressure [DBP], cardiac index [CI], and serum troponin elevation (defined as Tn I level ≥20 ng/L)), acid-base balance (pH, bicarbonate, lactate); gas exchange (P:F ratio and PₐCO₂); urine output and fluid balance; and the development of any thromboembolic event in hospital (deep vein thrombosis, pulmonary embolism).

Statistical analysis: Baseline characteristics were expressed as frequencies (n, %) means (standard deviation, SD), or medians (interquartile range, IQR), as dictated by data type. Between-group comparisons were performed using the chi-squared test, unpaired t-test, and Wilcoxon rank sum test, as appropriate. The mean peak serum creatinine level was compared by the unpaired t test; the incidence and stage of new or progressive AKI were explored using chi-squared test. Multilevel mixed effects linear regression analyses were used to assess serum creatinine trajectory, physiological and biochemical parameters. Competing risk regression analyses were used to examine time to liberation from CRRT (death was treated as a competing risk). Death was examined by the Kaplan-Meier method and logistic regression. Patients were examined by subgroups of age (dichotomized at 65 years), sepsis (yes/no), and prior exposure to RAAS inhibitors. Analyses were conducted using Stata MP16.0. A two-sided p value <0.05 was considered statistically significant.

Catecholamine exposure: Over the first 7 days, patients in the angiotensin II arm received less norepinephrine (305 vs 446 mcg per patient) and epinephrine (12 vs 31 mcg per patient) than patients in the control arm. Total vasopressin administration was higher in patients receiving angiotensin II than in control patients (0.35 vs 0.27 IU per patient) while metaraminol administration was similar between groups (80 vs 85 mcg per patient). Initiation of angiotensin II was associated with a reduction in catecholamine exposure over the first 24 hours, most marked in the first 6 hours of therapy. Time to vasopressor cessation was similar in patients who received angiotensin II and conventional vasopressors (45.5 vs 42.5 hours, p=0.84).

Renal Results: Peak serum creatinine level within the first 7 days was not different in patients receiving angiotensin II compared to matched controls (median 128 vs 126 μmol/L, p=0.78, Table 2). The median number of hours between the start of the intervention and the peak creatinine was 8.5 (4.5 to 37.5) in patients receiving angiotensin II compared to 9 (IQR 3 to 29) in the control group. There was no difference in the incidence of new or progressive AKI or in peak AKI stage at day 7. The trajectory of serum creatinine level over the first 7 days was similar between groups (β coefficient 2.96, 95% CI −24.64 to 30.55). There was no significant decrease in the risk of MAKE7 in patients who received angiotensin II compared to control (48% vs 30%, p=0.07). The incidence of new CRRT was similar, as was the time to new CRRT initiation. In patients who received CRRT, the time to liberation was not different (SHR 1.38, 95% CI 0.60 to 3.17).

Nonrenal Results: Median length of stay in ICU (median 3.5 vs 3.8 days, p=0.49) and hospital (13.8 vs 11.8 days, p=0.31) were not different in patients receiving angiotensin II or conventional vasopressors. ICU mortality was significantly lower in patients receiving angiotensin II, but the difference in hospital and 28-day mortality was not statistically significant (Table 2). Days alive and free of renal replacement therapy at 28- and 90-days were also not significantly different. However, the incidence of troponin elevations was lower in patients receiving angiotensin II. In terms of arterial and venous thromboembolic complications, the incidence of deep vein thrombosis and pulmonary embolism were similar. There were no significant differences between groups in terms of hemodynamic parameters, respiratory parameters, metabolic parameters, or fluid parameters.

TABLE 2
Clinical outcomes of critically ill patients receiving angiotensin II or conventional
vasopressors as first line therapy for the treatment of vasodilatory shock.

| | Angiotensin II | | Control | | |
|---|---|---|---|---|---|
| | (N = 40) | | (N = 80) | | P value |
| Primary outcome | | | | | |
| Peak creatinine at day 7 - median (IQR) | 128 | (89, 192) | 126 | (90, 176) | 0.78 |
| Peak creatinine at day 7 - mean (SD) | 154 | (87) | 147 | (78) | 0.67 |
| Secondary outcomes (renal) | | | | | |
| New or progressive AKI at day 7 - no. (%) | 23 | (66) | 49 | (70) | 0.66 |
| Peak AKI stage at day 7 - no. (%) | | | | | 0.39 |
| Nil | 12 | (34) | 21 | (30) | |
| Stage 1 | 9 | (26) | 24 | (34) | |
| Stage 2 | 5 | (14) | 15 | (21) | |
| Stage 3 | 9 | (26) | 10 | (14) | |
| New CRRT - no. (%) | 5 | (14) | 9 | (13) | 0.84 |
| Time to new CRRT, hours - median (IQR) | 38 | (31, 54) | 30 | (25,38) | 0.18 |
| Major adverse kidney event at 7 days - no. (%) | 12 | (30) | 38 | (48) | 0.07 |
| Secondary outcomes (mortality) | | | | | |
| ICU mortality - no. (%) | 4 | (10) | 21 | (26) | 0.04 |
| Hospital mortality - no. (%) | 8 | (20) | 24 | (30) | 0.24 |
| 28-day mortality - no. (%) | 7 | (18) | 23 | (29) | 0.18 |
| 90-day mortality - no. (%) | 9 | (22) | 24 | (30) | 0.39 |
| Days alive at 28 days - mean (SD) | 24.4 | (8.2) | 21.9 | (10.3) | 0.18 |
| Days alive at 90 days - mean (SD) | 73.8 | (32.0) | 65.4 | (38.1) | 0.23 |
| Days alive and RRT-free at 28 days - mean | 21.3 | (10.5) | 19.2 | (12.0) | 0.34 |
| (SD) | | | | | |
| Days alive and RRT-free at 90 days - mean | 69.8 | (32.8) | 61.8 | (39.8) | 0.28 |
| (SD) | | | | | |
| Secondary outcomes (safety) | | | | | |
| Deep vein thrombosis - no. (%) | 2 | (5) | 1 | (1) | 0.21 |
| Pulmonary embolus - no. (%) | 4 | (10) | 4 | (5) | 0.30 |
| Elevated serum troponin - no. (%) | 3 | (8) | 18 | (22) | 0.04 |

AKI, acute kidney injury; CRRT, continuous renal replacement therapy; ICU, intensive care unit; IQR, interquartile range; RRT, renal replacement therapy; SD, standard deviation. Elevated serum troponin was defined as a troponin I level ≥20 ng/L.

Subgroup analyses: The results of subgroup analyses are presented in Table 3. There were no qualitative differences in the primary outcome analysis across subgroups of age (dichotomized at 65 years) or cause of shock (sepsis vs non-sepsis). Patients on ACE inhibitors or angiotensin II receptor blockers prior to admission to hospital who received angiotensin II had a lower peak serum creatinine level compared to those receiving control vasopressors, while patients on no renin angiotensin aldosterone system inhibitors had a higher peak serum creatinine if they received angiotensin II compared to control (p value for interaction 0.03).

TABLE 3
Peak serum creatinine level (median, interquartile range) at day 7 in critically
ill patients receiving angiotensin II or conventional vasopressors as first
line therapy for the treatment of vasodilatory shock, according to subgroup

| | | Angiotensin II | | Control | | P value |
|---|---|---|---|---|---|---|
| | N | (N = 35) | | (N = 70) | | for interaction |
| Age, years | | | | | | 0.37 |
| <65 | 52 | 136 | (81 to 280) | 115 | (83 to 197) | |
| ≥65 | 53 | 139 | (117 to 241) | 155 | (123 to 226) | |
| Sepsis | | | | | | 0.93 |
| No | 47 | 123 | (89 to 236) | 123 | (83 to 160) | |
| Yes | 58 | 140 | (121 to 262) | 169 | (120 to 226) | |
| RAAS inhibition | | | | | | 0.03 |
| No | 71 | 158 | (115 to 280) | 126 | (85 to 186) | |
| ACE inhibitor | 12 | 121 | (78 to 200) | 183 | (129 to 232) | |
| Angiotensin receptor blocker | 22 | 125 | (95 to 194) | 160 | (126 to 293) | |

Key findings: Peak serum creatinine level over the first 7 days was not different in patients who received angiotensin II compared to conventional vasopressors. Creatinine trajectory, the incidence of AKI and MAKE7, and the requirement for and duration of CRRT were comparable. Although patients receiving angiotensin II had lower ICU mortality, 28-day and 90-day mortality were the same. The incidence of troponin elevation was lower in the angiotensin II group compared to the controls, but there were no differences in the rates of thromboembolic complications. Findings were not different according to age or presence of sepsis; patients with prior exposure to RAAS inhibitors who received angiotensin II had lower peak serum creatinine than those who received control vasopressors.

Serum Troponin Measurement: The incidence of elevated serum troponin in patients within the angiotensin II group was 3 subjects (8%, n=40), whereas the incidence of elevated serum troponin in patients within the control group was 18 (22%, n=80). This clinically meaningful difference was also statistically significant (P value of 0.04). This result demonstrates that administration of angiotensin II is effective to reduce incidence of elevated troponin levels in patients compared patients receiving conventional vasopressors (norepinephrine, vasopressin, metaraminol, epinephrine).

1. A method of treating cardiac injury in a subject comprising administering a therapeutically effective amount of angiotensin II to a subject in need thereof.

2. The method of claim 1, wherein the subject has cardiac injury.

3. The method of claim 1, wherein the subject has increased blood troponin.

4. (canceled)

5. The method of claim 3, wherein the blood troponin is Troponin I.

6. The method of claim 3, wherein increased blood troponin is blood troponin levels above the 99th percentile upper reference limit.

7. (canceled)

8. The method of claim 1, wherein the therapeutically effective amount of angiotensin II prevents further progression of the cardiac injury.

9. The method of claim 1, wherein the subject does not have cardiac injury.

10. The method of claim 1, wherein the administration of angiotensin II allows for reduction of the amount of catecholamine and/or vasopressin needed to be administered to the subject.

11. The method of claim 9, wherein the therapeutically effective amount of angiotensin II prevents cardiac injury.

12. A method of treating catecholamine-associated or vasopressin-associated cardiac injury in a subject comprising administering angiotensin II to a subject in need thereof.

13. The method of claim 12, wherein the subject has cardiac injury.

14. The method of claim 13, wherein the subject has increased blood troponin.

15.-17. (canceled)

18. The method of claim 12, wherein the administration of angiotensin II is effective to prevent increase in blood troponin.

19. The method of claim 12, wherein the subject is taking or is in need of taking a catecholamine, vasopressin, or a combination thereof.

20. The method of claim 19, wherein the administration of angiotensin II allows for reduction of the amount of catecholamine and/or vasopressin being administered.

21. The method of claim 19, wherein the administration of angiotensin II allows for discontinuation of the administration of catecholamine and/or vasopressin.

22. The method of claim 12, wherein the therapeutically effective amount of angiotensin II prevents further progression of the cardiac injury.

23. The method of claim 12, wherein the subject does not have cardiac injury.

24. (canceled)

25. The method of claim 23, wherein the therapeutically effective amount of angiotensin II prevents catecholamine-associated and/or vasopressin-associated cardiac injury.

26. The method of claim 23, wherein the administration of angiotensin II allows for reduction of the amount of catecholamine and/or vasopressin needing to be administered to the subject.

27. The method of claim 12, wherein the subject has been previously administered a catecholamine and/or vasopressin, comprising the steps of (1) administering angiotensin II to the subject and (2) discontinuing administration of the catecholamine and/or vasopressin.

28. The method of claim 12, wherein the subject is receiving catecholamine and/or vasopressin at an initial dose, comprising the steps of (1) administering angiotensin II to the subject and (2) administering catecholamine and/or vasopressin to the subject at a dose that is lower than the initial dose.

29.-37. (canceled)

38. The method of claim 1, further comprising measuring the amount of troponin present in a sample obtained from the subject before administering angiotensin II.

39. The method of claim 1, further comprising (1) measuring the amount of troponin present in a sample obtained from the subject before administering angiotensin II and (2) measuring the amount of troponin present in a sample obtained from the subject after administering angiotensin II.

40. (canceled)

41. The method of claim 1, wherein the administration of angiotensin II allows for reduction in the amount of catecholamine and/or vasopressin being administered such that there is a clinically significant reduction in blood troponin.

42.-46. (canceled)

47. The method of claim 1, wherein angiotensin II is administered at a rate of about 1 ng/kg/min to 20 ng/kg/min.

48. (canceled)

49. The method of claim 1, wherein the administration of angiotensin II is effective to raise the blood pressure of the subject to a mean arterial pressure (MAP) of about 65 mm Hg or above.

50. The method of claim 1, wherein the administration of angiotensin II is effective to raise the blood pressure of the subject to a mean arterial pressure (MAP) above about 65 mm Hg, and which is effective to reduce the dose of the catecholamine and/or vasopressin required to maintain a MAP above about 65 mm Hg to the equivalent of less than about 0.05-0.10 mcg/kg/min NED.


SEQUENCE LISTING (4 sequences)

SEQ ID NO: 1 - protein, 8 aa, synthetic construct
DRVYIHPF

SEQ ID NO: 2 - protein, 8 aa, synthetic construct
DRVYVHPF

SEQ ID NO: 3 - protein, 6 aa, synthetic construct
VYIHPF

SEQ ID NO: 4 - protein, 9 aa, synthetic construct
NRVYYVHPF